(19) United States
(12) Plant Patent Application Publication
(10) Pub. No.: US 2003/0005499 P1
(43) Pub. Date: Jan. 2, 2003

(54) Peach tree named Island Princess

(76) Inventors: Alan R. Asdoorian, Kingsburg, CA (US); Lora A. Asdoorian, Kingsburg, CA (US)

Correspondence Address: MARK D MILLER, KIMBLE, MACMICHAEL & UPTON, 5260 NORTH PALM AVENUE, SUITE 221, FRESNO, CA, 93704, US

(21) Appl. No.: 09/895,610

(22) Filed: Jun. 29, 2001

(51) Int. Cl. A01H 5/00

(52) U.S. Cl. PLT/197

(57) ABSTRACT

A new and distinct variety of peach tree which is somewhat similar to the ‘Island King’ peach tree (U.S. Plant Pat. No. 9199) from it is a sport bud from which it is distinguished by producing a later ripening fruit approximately six to eight days after the fruit produced by the ‘Island King’ peach tree.

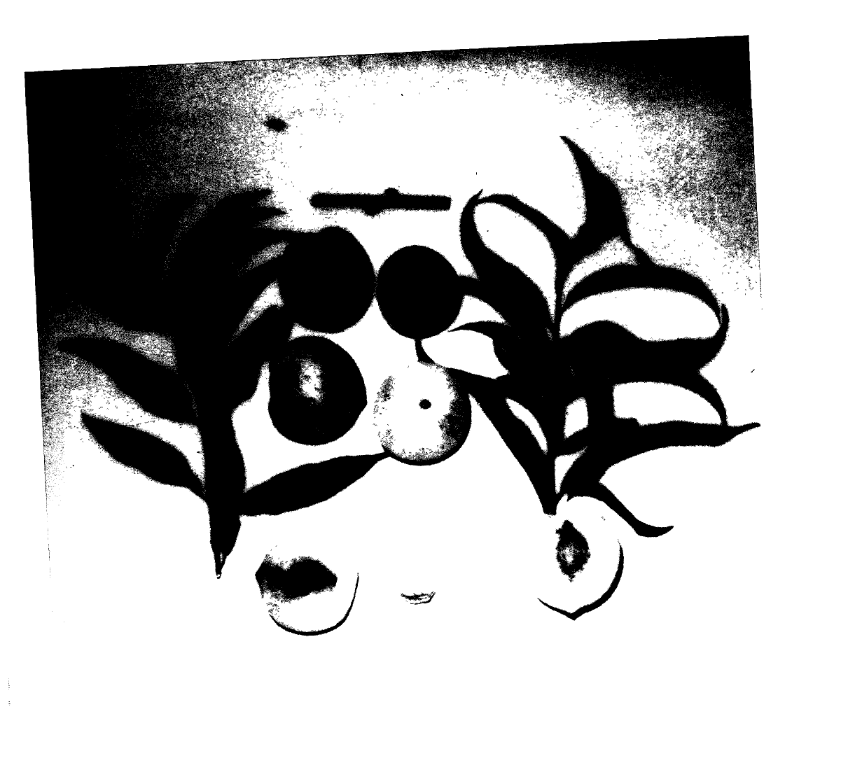

### BACKGROUND OF THE NEW VARIETY

This present invention refers to a new variety of peach which will hereinafter be denominated as the ‘Island Princess’ peach tree which is a clingstone fruit which is mature for commercial harvesting and shipment May 1-3 in a normal spring year in the San Joaquin Valley of Central California as an early market peach with a good red blush coloration.

In the development of new commercial varieties specific characteristics provide a premium on those fruit varieties which are early or late maturing in the growing season. However, particular early varieties have small size, lack of flavor, or coloration. In some instances there are other undesirable characteristics that decrease the commercial success, such as poor holding or storing capabilities. Therefore, in order for a fruit to be a commercial success, it must be of good size, good color, good flavor, beside good holding capabilities. At the same time the date of maturing must be different from other similar fruit. This new invention meets all of the aforementioned criteria and therefore is of commercial appeal to the consumer.

### ORIGIN AND ASEXUAL REPRODUCTION OF THE NEW VARIETY

This present variety was discovered by the inventor in his four acre orchard of ‘Island King’ peach trees (U.S. Plant Pat. No. 9199) which is located near Kingsburg, Calif., in the San Joaquin Valley of Central California. The invention was first discovered as a sport of the ‘Island King’ tree in 1998. The new variety was first asexually reproduced in the dormant season of 1999/2000 by bud grafting onto 600 Nemaguard Rootstocks on the owner's property, adjacent to the original peach orchard. The asexually reproduced trees first bore fruit of the new variety in May 2001. The inventor carefully compared the asexually reproduced trees with the parent sport, including the fruit and the parent, and the progeny are in all respects identical to the parent tree.

### SUMMATY OF THE NEW VARIETY

The ‘Island Princess’ peach tree is characterized by producing a large clingstone fruit which has good blush coloration and is ripe for commercial harvesting and shipment approximately May 1-3 in the San Joaquin Valley of Central California. The new variety is most similar to ‘Island King’ peach tree (U.S. Plant Pat. No. 9199) from which it is a sport but from which it is distinguishable in that it ripens six to eight days later than the fruit of the ‘Island King’ peach tree. The fruit of this new variety possesses a very good flavor with a pleasant aroma which makes it greatly acceptable for an early ripening variety.

### BRIEF DESCRIPTION OF THE DRAWING

The accompanying drawing is a color photograph displaying fruit, foliage, and a piece of second year branch at the top of the photograph. Foliage of the upper side of the leaves are on the left side of the photograph while foliage of the underside of the leaves are on the right side; four whole fruit are in the center with the upper left displaying the suture, the upper right displaying the blossom end of the fruit, the lower left fruit shows a side view with the cheek surface and the lower right the distal end of the fruit. At the bottom a fruit is cut in half with the left side showing the flesh and pit well, a pit in the center with a half of the fruit showing the flesh and pit.

### DETAILED DESCRIPTION

Referring more specifically to the pomological description of this new and distinct variety of peach tree, the following details have been observed under the ecological conditions prevailing in the orchard of origin which is located near Kingsburg, Calif. in the San Joaquin Valley of Central California. All major color code designations are by reference to Dictionary of Color by Maerz and Paul, First Edition, 1930. Common color names are also occasionally employed.

### TREE

Size: Small to medium in height and width

Vigor: Early indicates that vigor is good (40-49 inches of new growth this growing season).

Figure (form): Open and spreading with open vase system of training. Productivity is good for tree of this age. Normal regular bearing (i.e., every year, not in alternate years).

Trunk size: At level above the graft union, approximately 10-12″ above soil level. Circumference is 1.875 inch-42.61 mm.

Color.—Beaver (15-A-6)

Branches: Size—Medium Circumference 1.0625 inches (16.74-26.99 mm)

a. Mature.—slightly rough

b. Immature.—smooth Color code (one year or older), Horse chestnut (8-J-5) Color code (immature), near Calliste green (19-L-7) Three to four scaffold with lateral branches, at this stage of growth, developing fruitwood and allow sunlight for fruit colors

### LEAVES

Size: Medium to large

Length: 135 mm-151 mm (5.31-5.94 inches)

Width: 32 mm-38 mm (1.26-1.50 inches)

Color Code:

Upper surface.—Mt. Vernon Green (23-J-8)

Lower surface.—Art Green (22-L-7)

Marginal form: Crenate

Leaf vein:

Color code.—Shadow Green (20-J-3)

Thickness.—1 mm

Glandular characteristics—reniform.—2.—opposite sides of leaf at the base

Petiole:

Size.—Medium

Length.—10 mm-12 mm (0.37-0.37 inch)

Diameter.—2 mm (0.08 inch)

Color code.—Shadow green (20-J-3)

### FLOWER

Flower buds: Hardy under typical San Joaquin Valley, Central California climate conditions.

Size.—Medium

Diameter.—5 inch (6 mm)

Length.—10.3 mm (41 inch)

Form.—conic shape

Bud Scales:

Color: Near Maroon near sepals (7-L-7)

Generally.—Showy type

Date of Bloom: approximately 15-20% by Feb. 28, 2001 with full bloom by March 10^{th}.

Size of flowers: Medium to large

Diameter: when fully expanded—43 mm-45 mm (1.67-1.77 inches)

Bloom quality: abundant

Petals:

Size.—Medium to large

Length.—16-18 mm

Width.—15-18 mm

Form.—broadly ovate

Number.—5

Color: from light pink (1-C-5) to Pink older petals (1-D-1)

Petal claws: broadly truncate

Width.—1 mm (0.039 inch)

Length.—1.5 mm (0.059 inch)

Petal margins: moderately undulated with rounded edges.

Flower pedicel: very short, Aug 2 mm (0.08 inch)

Color.—Lily Green (19-A-5)

Surface.—pubescent

Sepals:

Size.—Medium to large, 6.3 mm (0.25 inch)

Color.—Amaranth (44- L-1)

Number.—5

Anthers:

Size.—small

Color.—Mineral orange (6-J-3) Center is Straw (10-F-2)

Stamens: 10-18 mm (0.39-0.71 inch)

Filament color.—very Light Pink (1-B-1)

Number.—35-45

Pistil:

Length.—18 mm (0.71 inch)

Color.—near Kildare Green (20-J-5)

Surface.—pubescent

Number.—One

### FRUIT

Date of maturity: May 1-3 in the San Joaquin Valley of Central California

Size:

Diameter axial plane.—58 mm-64 mm (2.28-2.54 inch)

Transverse in suture plane.—60 mm-66 mm (2.36 inch-2.60 inch)

Transverse at rt. angle to suture plane.—62 mm-65 mm (2.44 inch 2.68 inch)

Form:

Uniform.—

Symmetrical or Asymmetrical.—Shape of fruit is slightly asymmetrical

Suture: Shallow with Yellow coloration from stem end to apex

Ventral Surface: uneven

Stem Cavity:

Width.—20 mm (0.79 inch)

Depth.—12 mm (0.47 inch)

Length.—21 mm (0.83 inch)

Stem: Short

Diameter.—0.39 inch-0.47 inch

Caliper.—6 mm-8 mm (0.24-0.31 inch)

Apex: Slightly Rounded

Pistil point: Oblique

Skin: Thickness normal for peach, slightly pubescent

Texture: Firm

Tendency: None observed

Color Code:

Blush color.—Chalet R (6-K-10)—over 50% of fruit

Ground color.—Corn -(10-J-5)

Flesh color.—Apricot Y (9-K-5)

Color of surface of pit cavity.—Apricot (9-K-5)

Color of pit well.—Apricot (9-K-5)

Juice Production: Moderate

Aroma: Good

Texture: Firm

Fibers:

Number.—Few

Texture.—Firm

Ripening: Even

Eating quality: Very good

Stone:

Attachment.—Clingstone

Fibers.—Few, moderate in length

Size.—Length—33 mm-38 mm (1.29-1.50 inch) Width—23 mm-26 mm (0.91-1.02 inch) Thickness—16 mm-21 mm (0.63-0.83 inch)

Form: Ovate

Apex: Sharply acute

Color Code, when dry: Toast (13-E-8)

Base: Somewhat ovate in shape

Sides: Unequal

Ridges: On both sides of stone with narrow ridge on ventral side

Tendency to split: None observed

Use: Fresh Market The above description of this new variety of peach tree is based on growing conditions prevailing near Kingsburg, Calif. in the Central San Joaquin Valley of California. Variations of the usual magnitude and characteristics may occur due to changes in cultural practices including irrigation, fertilization, pruning practices, and changes in the weather conditions.

1. a new and distinct variety of peach tree as described and illustrated which is somewhat similar to ‘island king’ (united states plant patent 9199) from which it is a bud sport but from which it is distinguished by producing fruit that ripens 6-8 days after ‘island king’ with good exterior color and eating quality which are mature for commercial harvesting and shipment approximately may 1 to 3 in a normal year.
